(19) United States
(12) Patent Application Publication
(10) Pub. No.: US 2018/0146887 A1
(43) Pub. Date: May 31, 2018

(54) Respiration Detection Device

(71) Applicant: Huang-Chung Cheng, Tainan City (TW)

(72) Inventor: Huang-Chung Cheng, Tainan City (TW)

(21) Appl. No.: 15/361,523

(22) Filed: Nov. 28, 2016

(51) Int. Cl. A61B 5/08; A61B 5/00; A63B 71/08

(52) U.S. Cl. CPC A61B 5/0816; A61B 5/682; A63B 71/085; A61B 5/7278; A61B 2503/10; A61B 5/742; A61B 5/486; A61B 5/746; A61B 2562/0204; A61B 5/0022

(57) ABSTRACT

A respiration detection device includes a mouth guard mounted in a mouth of a user. A coupling seat is mounted to a front end of the mouth guard and is located in a position in front of a nose and the mouth of the user. A detection unit includes a circuit board, a microphone, a micro-processing circuit, and a transmission circuit. The circuit board is mounted to the coupling seat and is connected to a battery. The micro-processing circuit, the transmission circuit, and the microphone are mounted on the circuit board. The micro-processing circuit is configured to receive and process a message from the microphone to indicate a respiratory rate of the user. The transmission circuit is electrically connected to the micro-processing circuit and is configured to proceed with data transmission with an external device. The respiration detection device can accurately detect the respiratory rate in real time.

### BACKGROUND OF THE INVENTION

The present invention relates to a respiration detection device and, more particularly, to a detection device for accurately detecting the respiratory rate.

Respiration provides the body with oxygen and removes the waste gas. The respiration rate is one of the indexes for detecting the cardiopulmonary function of the body. An adult at rest breathes about 12-20 times per minute. The body needs more oxygen during exercise, and the respiratory rate increases. The respiratory rates are different due to different exercise intensities. For example, the respiratory rate during fast running is greater than slow running. Thus, timely detection of the respiratory rate can avoid danger resulting from excessive exercise during body abnormality.

Although people can detect the respiratory rate by themselves, real-time detection during running and cycling are difficult. Namely, the respiratory rate during exercise cannot be known.

U.S. Patent Publication No. 2012/0172677 discloses a monitoring system for monitoring data of a user. In an example, the monitoring system includes a dental appliance (such as a mouth guard) mounted in a mouth of the user. A circuit board and a sensor are mounted in the mouth guard for detection of the respiratory rate or other biometric data. However, the mouth guard in the mouth is not in a position corresponding to the respiratory tract, and the detected state cannot be displayed to the outside. The detected respiratory rate could have a larger error, and the breathing state cannot be known in real time.

### BRIEF SUMMARY OF THE INVENTION

An objective of the present invention is to provide a detection device for detecting respiratory rate with improved accuracy.

Another objective of the present invention is to provide a detection device for providing real time information of the breathing state.

A respiration detection device according to the present invention includes a mouth guard, a coupling seat, and a detection unit. The mouth guard is adapted to be mounted in a mouth of a user. The mouth guard includes a front end having a first coupling portion. The coupling seat is mounted to the front end of the mouth guard and is adapted to be located in a position in front of a nose and the mouth of the user. The coupling seat includes a second coupling portion coupled with the first coupling portion. The detection unit includes a circuit board, a microphone, a micro-processing circuit, and a transmission circuit. The circuit board is mounted to the coupling seat and is adapted to be connected to a battery. The micro-processing circuit, the transmission circuit, and the microphone are mounted on the circuit board. The micro-processing circuit is configured to receive and process a message from the microphone to indicate a respiratory rate of the user. The transmission circuit is electrically connected to the micro-processing circuit and is configured to proceed with data transmission with an external device.

The respiration detection device as claimed in claim 1, with the detection unit further including a plurality of indication lamps electrically connected to the micro-processing circuit, wherein when the micro-processing circuit receives a message indicating the breathing times reach a set value, the micro-processing circuit activates a corresponding one of the plurality of indication lamps to emit light.

In an example, the transmission circuit includes a blue tooth module configured to proceed with data transmission with a blue tooth module of the external device.

In an example, the first coupling portion of the mouth guard has a slot, and the second coupling portion of the coupling seat has a tab inserted into the slot.

In an example, the coupling seat includes a front wall, a rear wall, and an annular wall. The front wall, the rear wall, and the annular wall define a chamber. The rear wall includes a detection hole. The circuit board of the detection unit is mounted in the chamber of the coupling seat. The microphone is aligned with the detection hole of the coupling seat.

In another example, the coupling seat includes a front wall, a rear wall, and an annular wall. The front wall, the rear wall, and the annular wall define a chamber. The rear wall includes a detection hole and a plurality of through-holes. The annular wall includes an upper end having a plurality of holes. The detection unit further includes a plurality of indication lamps. The circuit board is mounted in the chamber of the coupling seat. A power switch and a power indication lamp are mounted in the plurality of through-holes. The microphone is aligned with the detection hole of the coupling seat. The plurality of indication lamps is mounted in the plurality of holes of the annular wall of the coupling seat.

The mouth guard is placed into a mouth of a user with the rear wall of the coupling seat located in a position in front of a nose and the mouth of the user. The three indication lamps are set to emit in accordance with different respiration rates. An application (APP) of the present invention can be downloaded into the external device with a blue tooth module. The BMI of the user is used to obtain the maximum breathing times per minute of the user. The breathing times per minute of the user can be displayed. Furthermore, the user can change settings to make each of the indication lamps to emit light according to the corresponding respiratory rate.

In practice, the sound resulting from breathing through the nose and mouth of the user can be directly received by the microphone through the detection hole, providing excellent detection accuracy. The signal received by the microphone is inputted to the micro-processing circuit. The user uses the external device to wirelessly obtain the detection value of the micro-processing circuit via the transmission circuit. The user during exercise can know whether the current respiratory rate is normal in real time and can maintain his or her health by understanding his or her physical condition.

The present invention will become clearer in light of the following detailed description of illustrative embodiments of this invention described in connection with the drawings.

### DESCRIPTION OF THE DRAWINGS

FIG. 1 is an exploded, perspective view of a respiration detection device according to the present invention.

FIG. 2 is a perspective view of the respiration detection device according to the present invention.

FIG. 3 is a cross sectional view of the respiration detection device according to the present invention.

FIG. 4 is a diagrammatic block diagram of a detection unit of the respiration detection device according to the present invention.

FIG. 5 is a diagrammatic perspective view illustrating use of the respiration detection device according to the present invention.

### DETAILED DESCRIPTION OF THE INVENTION

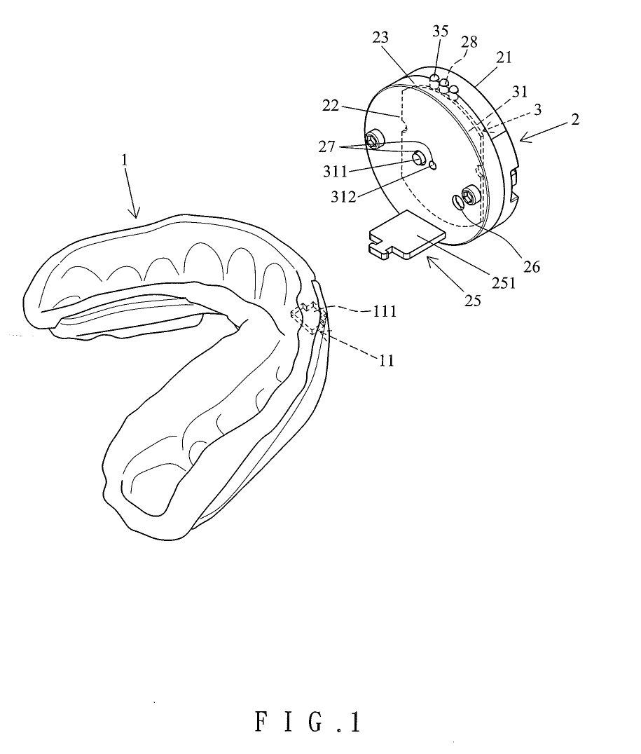

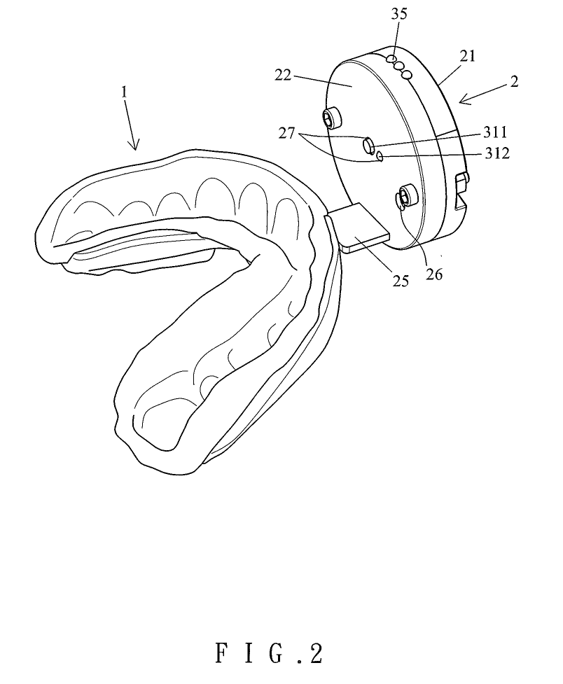

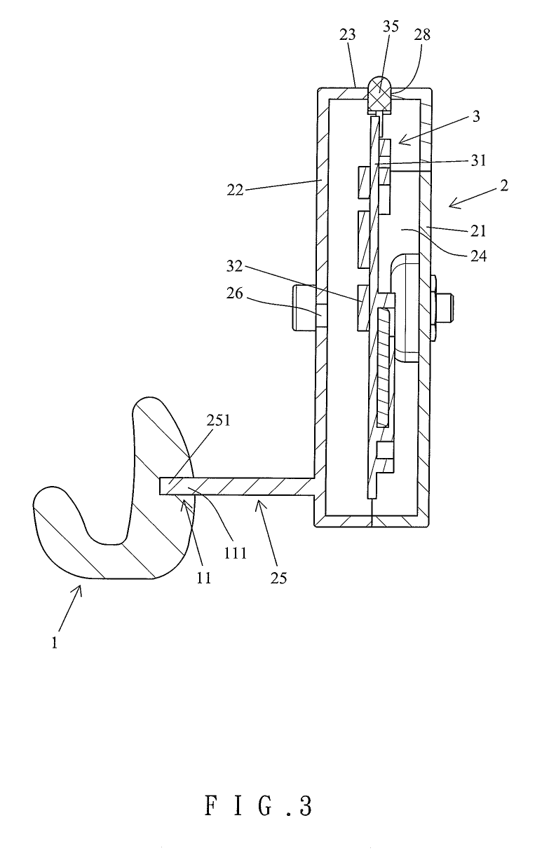

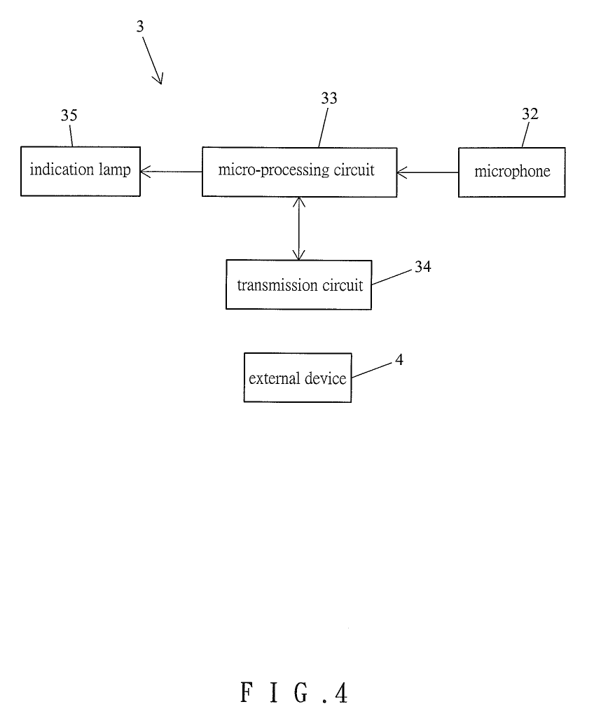

With reference to FIGS. 1-4, a respiration detection device according to the present invention includes a mouth guard 1, a coupling seat 2, and a detection unit 3. The mouth guard 1 is adapted to be mounted in a mouth of a user. The mouth guard 1 includes a front end having a first coupling portion 11. The first coupling portion 11 includes a slot 111.

The coupling seat 2 is mounted to the front end of the mouth guard 1 and includes a front wall 21, a rear wall 22, and an annular wall 23. The front wall 21, the rear wall 22, and the annular wall 23 define a chamber 24. A second coupling portion 25 protrudes rearward from a lower end of the annular wall 22 and is coupled to the first coupling portion 11. The second coupling portion 25 has a tab 251 inserted into the slot 111. The rear wall 22 includes a detection hole 26 and a plurality of through-holes 27. The annular wall 23 includes an upper end having three holes 28.

The detection unit 3 includes a circuit board 31, a microphone 32, a micro-processing circuit 33, a transmission circuit 34, and three indication lamps 35. The circuit board 31 is mounted in the chamber 24 of the coupling seat 2 and is connected to a battery (not shown). A power switch 311 and a power indication lamp 312 are mounted in the through-holes 27 of the rear wall 22 of the coupling seat 2. The micro-processing circuit 33, the transmission circuit 34, and the microphone 32 are mounted on the circuit board 31. The microphone 32 is aligned with the detection hole 26 of the coupling seat 2. The micro-processing circuit 33 is configured to receive and process a message from the microphone 32 to indicate a respiratory rate of the user. The transmission circuit 34 is electrically connected to the micro-processing circuit 33 and can include a blue tooth module configured to proceed with data transmission with a blue tooth module of an external device 4 (such as a mobile phone or any other device). The indication lamps 35 can be light emitting diodes (LEDs), are mounted in the holes 28 of the annular wall 23 of the coupling seat 2, and are electrically connected to the micro-processing circuit 33. The indication lamps 35 are different and are set to correspond to different breathing times. Furthermore, when the micro-processing circuit 33 receives a message indicating the breathing times reach a set value, the micro-processing circuit 33 activates a corresponding one of the indication lamps 35 to emit light.

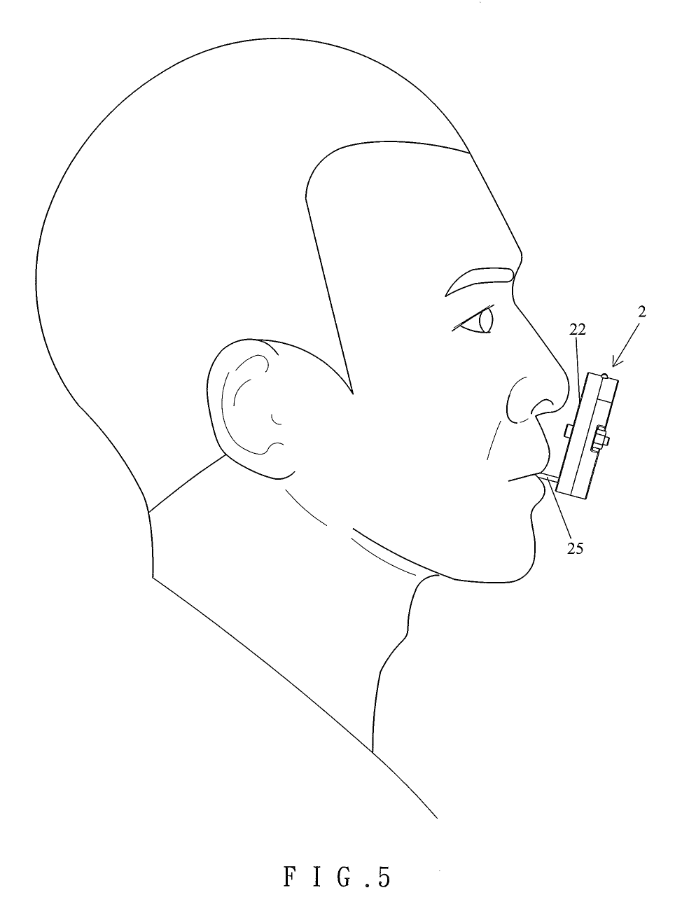

With reference to FIGS. 1-5, the tab 251 of the second coupling portion 25 of the coupling seat 2 is inserted into the slot 111 of the first coupling portion 11 of the mouth guard 1. The mouth guard 1 is placed into a mouth of a user with the rear wall 22 of the coupling seat 2 located in a position in front of a nose and the mouth of the user. The three indication lamps 35 are set to emit light in accordance with different respiration rates. For example, flickering green, orange, and red lights respectively corresponding to 50%, 70%, and 90% of the maximum breathing times can be emitted to remind the user of his or her current physical condition in real time.

An application (APP) of the present invention can be downloaded into the external device 4 with a blue tooth module. By using the APP, the sex, age, height, and weight of the user can be inputted and converted into the body mass index (BMI). The BMI is multiplied with a preset coefficient to obtain the maximum breathing times per minute of the user. The breathing times per minute of the user can be displayed. Furthermore, an alarm effect can be provided by the different indication lamps 35 respectively corresponding to 50%, 70%, and 90% of the maximum breathing times. Since different users have different physical conditions, and the APP can be finely adjusted, such that an alarm effect can be provided at different set values of different respiratory rates through the transmission circuit 34 and the micro-processing circuit 33. Furthermore, the user can change the set values to make each of the indication lamps 35 to emit light according to the corresponding respiratory rate, thereby avoiding danger resulting from excessive training of an athlete.

In practice, the sound resulting from breathing through the nose and mouth of the user can be directly received by the microphone 32 through the detection hole 25, providing excellent detection accuracy. The signal received by the microphone 32 is inputted to the micro-processing circuit 33. The user uses the external device 4 to wirelessly obtain the detection value of the micro-processing circuit 33 via the transmission circuit 34. The user during exercise can know whether the current respiratory rate is normal in real time and can maintain his or her health by understanding his or her physical condition.

Thus, the present invention can accurately detect the respiratory rate and can display the effect in real time. As a result, the present invention is suitable for various athletes.

Although specific embodiments have been illustrated and described, numerous modifications and variations are still possible without departing from the scope of the invention. The scope of the invention is limited by the accompanying claims.

1. A respiration detection device comprising:
a mouth guard adapted to be mounted in a mouth of a user, with the mouth guard including a front end having a first coupling portion;
a coupling seat mounted to the front end of the mouth guard and adapted to be located in a position in front of a nose and the mouth of the user, with the coupling seat including a second coupling portion coupled with the first coupling portion; and
a detection unit including a circuit board, a microphone, a micro-processing circuit, and a transmission circuit, with the circuit board mounted to the coupling seat and adapted to be connected to a battery, with the micro-processing circuit, the transmission circuit, and the microphone mounted on the circuit board, with the micro-processing circuit configured to receive and process a message from the microphone to indicate a respiratory rate of the user, and with the transmission circuit electrically connected to the micro-processing circuit and configured to proceed with data transmission with an external device.

2. The respiration detection device as claimed in claim 1, with the detection unit further including a plurality of indication lamps electrically connected to the micro-processing circuit, wherein when the micro-processing circuit receives a message indicating the breathing times reach a set value, the micro-processing circuit activates a corresponding one of the plurality of indication lamps to emit light.

3. The respiration detection device as claimed in claim 2, wherein the transmission circuit includes a blue tooth module configured to proceed with data transmission with a blue tooth module of the external device.

4. The respiration detection device as claimed in claim 1, wherein the first coupling portion of the mouth guard has a slot, and the second coupling portion of the coupling seat has a tab inserted into the slot.

5. The respiration detection device as claimed in claim 1, with the coupling seat including a front wall, a rear wall, and an annular wall, with the front wall, the rear wall, and the annular wall defining a chamber, with the rear wall including a detection hole, with the circuit board of the detection unit mounted in the chamber of the coupling seat, and with the microphone aligned with the detection hole of the coupling seat.

6. The respiration detection device as claimed in claim 1, with the coupling seat including a front wall, a rear wall, and an annular wall, with the front wall, the rear wall, and the annular wall defining a chamber, with the rear wall including a detection hole and a plurality of through-holes, with the annular wall including an upper end having a plurality of holes, with the detection unit further including a plurality of indication lamps, with the circuit board mounted in the chamber of the coupling seat, with a power switch and a power indication lamp mounted in the plurality of through-holes, with the microphone aligned with the detection hole of the coupling seat, and with the plurality of indication lamps mounted in the plurality of holes of the annular wall of the coupling seat.
